(19) United States
(12) Patent Application Publication
(10) Pub. No.: US 2021/0030978 A1
(43) Pub. Date: Feb. 4, 2021

(54) CARTRIDGE WITH SINGLE-LAYER CONTAINER AND ITS NOZZLE-SHAPED CAP FOR NEBULIZATION INHALATION

(71) Applicants: Cai Gu Huang, Shrewsbury, MA (US); Jian Cai, Shanghai (CN)

(72) Inventors: Cai Gu Huang, Shrewsbury, MA (US); Jian Cai, Shanghai (CN)

(73) Assignee: Cai Gu Huang, Shrewsbury, MA (US)

(21) Appl. No.: 16/941,974

(22) Filed: Jul. 29, 2020

Related U.S. Application Data
(60) Provisional application No. 62/879,549, filed on Jul. 29, 2019.

(51) Int. Cl. A61M 15/00; A61M 11/00

(52) U.S. Cl. CPC A61M 15/009; A61M 15/0025; A61M 2205/121; A61M 2207/00; A61M 2205/123; A61M 11/006

(57) ABSTRACT

The present invention is directed to a single-layer cartridge, and a process for producing a plastic single-layer container included in the cartridge. The single-layer cartridge comprises a plastic single-layer container, a seal and a nozzle-shaped cap or stopper, wherein the single-layer cartridge can be used to connected with a soft mist inhalation device or inhalation atomizer.

### PRIORITY STATEMENT

This application claims the benefit of U.S. Provisional Patent Application No. 62/879,549, filed on Jul. 29, 2019, the contents of which are incorporated herein by reference in its entirety.

### BACKGROUND OF THE INVENTION

A double layer cartridge containing a plastic container having a very thin and flexible inner layer and a hard, metallic outer layer was disclosed in US 2001009151A, which is used with a soft mist inhalation device. The double layer plastic container is difficult and expensive to make and suffers from poor control during the plastic blow molding process.

Furthermore, it is desirable to connect a pump to the single-layer cartridge so that the pharmaceutical formulation can be accurately expelled through a suitable atomizer to generate a soft mist for soft mist inhalation. A suitable atomizer with a pump can obtain pressure equalization so as to allow the solution to be pumped out. The linkage between cartridge and the inhalation device can be in the form of plug-in type, screw type or a bayonet type connection. In an embodiment, the linkage is in the form of non-releasable connection. The single layer cartridge of the current invention may be connected to or/in combination with a soft mist inhalation device and/or an inhalation atomizer comprising a block function, a counter, a nozzle system and a pressure generator such as those disclosed in patent application US20190030268 “inhalation atomizer comprising a blocking function and a counter”. It has now been surprisingly found that a single-layer suitably dimensioned plastic container is suitable for use as the cartridge for a soft mist inhalation device. The single layer plastic container is much easier and less expensive to manufacture.

### SUMMARY OF THE INVENTION

The present invention relates to a single-layer cartridge and a process for producing the container included in the cartridge. The single-layer cartridge comprises a plastic single-layer container, a seal, an opening, a transfer bed, and a nozzle-shaped cap or stopper, wherein the single-layer cartridge can be used to connect with a soft mist inhalation device or inhalation atomizer. The single-layer cartridge may be fitted into and/or protected by a metallic or aluminum canister.

The invention further provides a process for making the single-layer cartridge, wherein injection molding is used to form a preform and then the preform is blow molded. Furthermore, the process allows for including a pressure equalization opening in the single-layer container without other disadvantages. The present invention further provides a cartridge with a nozzle-shaped cap or a stopper, which is fixed by snap-in process to the container neck. The container further comprises a transfer bed in between the container neck and nozzle-shaped cap or stopper, which is provides support to bear the force during the snap-in process.

One aspect of the current invention is a single-layer cartridge and its production process wherein pressure equalization is maintained through the neck-based dispose. The injection-blow-molded single-layer plastic container is adapted to be connected to the cap or stopper, and inserted into an aluminum canister. The pump process equalizes pressure at the snapped-in conjunction without endangering the integrity of the plastic container to achieve better liquid withdrawal and to reduce waste.

According to another aspect of the current invention, there is a process for making the single-layer cartridge, wherein injection molding is used to form a semi-finished product or preform, and then a blow molding process at a low temperature is used to provide the single-layer cartridge. During the injection molding process the temperature is between about 230 and about 270 degrees C. During the blow molding process the temperature is between about 10 and about 15 degrees C.

In an embodiment, the single-layer plastic container length is about 20 to about 70 mm, preferably about 40 to about 55 mm, most preferably about 45 to about 50 mm. In an embodiment, the single-layer container thickness is about 0.05 mm to about 1.0 mm, preferably about 0.1 mm to about 0.3 mm.

Once the nozzle-shaped cap or stopper is snapped onto the single layer plastic container, it cannot detach from the single layer plastic container. An inverted sharp edged surface of the snap-in tab engages a coordinated snap-in groove on the upper part of the container. The inverted sharp-edged surface results in the generation of a lock and high snap-in force. The single layer plastic container has a transfer bed at the center of the container neck. The transfer bed is rigid and fixed and provides support during the snap-in process.

The single layer cartridge of the current invention can be connected to a soft mist inhalation device to draw-off or dispense a liquid pharmaceutical formulation that comprises a pharmaceutical active substance. The nozzle-shaped cap or stopper is connected to the single-layer container and can take the form of a sealed closing with an axially centered guide for drawing liquid through a connection part. The stopper is non-releasably connected by a rigid metallic or aluminum canister which is hard and has a stable shape. In one embodiment, the stopper preferably contains a thermoplastic material and is connected to the plastic single-layer container.

The connection of the single-layer cartridge to the dispensing device can be in the form of push plug-in connection, or a bayonet connection and is preferably a releasable connection. Preferably, the dispensing device is an inhalation atomizer or a soft mist inhalation device. In one embodiment, the connection comprises a block function through a locking mechanism, a counter, a nozzle system, and a pressure generator along with a drive spring, wherein the number of actuations of the device can be counted by a mechanical counter. In one embodiment, the stopper is provided in the insertion connection part with a funnel shaped centered guide means for the dispensing or draw-off linking part of the soft mist inhalation device. The stopper or nozzle-shaped cap can be a covering that connects to the draw-off linking part or can be in the form of press fit for the latter. In one embodiment, after insertion, the end of the insertion part is near the bottom of the container, is closed by an outlet seal, which can be arranged relative to the axis of the insertion connection part. The outlet seal can be pierced when the draw-off linking part is entered into the cartridge. The outlet seal prevents the liquid from escaping into the insertion connection part during storage of the cartridge.

A sealing disc provided with sealing beads or ridges can be positioned between the upper edge of the container and the stopper and is stable with respect to shape and inside of the stopper. The sealing disc also prevents the penetration of dirt below the disc and diffusion of liquid during storage of cartridge.

### BRIEF DESCRIPTION OF THE DRAWINGS

FIG. 1 shows a complete overview of the single-layer cartridge.

FIG. 2 shows a schematic presentation of the single-layer plastic container with cross section in the direction of the container central axis through the base area as an embodiment of the container.

FIG. 3 shows a schematic representation of a cross-section view of the nozzle-shaped stopper of the container through the central axis

FIG. 4 shows a schematic representation of a top view of the nozzle-shaped stopper.

FIG. 5 shows a cross section view of the soft silicon type material disc.

FIG. 6 shows a schematic top view of the soft silicon type material disc.

FIG. 7 shows a compressed negative pressure effect of the single-layer cartridge.

FIG. 8 shows a schematic section of a typical soft mist inhalation device.

The use of identical or similar reference numerals in different figures denotes identical or similar features.

### DETAILED DESCRIPTION OF THE INVENTION

The present invention relates to a single-layer cartridge, and a process for producing a plastic single-layer container included in the cartridge. The single-layer cartridge comprises a plastic single-layer container, a seal and a nozzle-shaped cap or stopper, wherein the single-layer cartridge can be connected to a soft mist inhalation device or inhalation atomizer. The single-layer cartridge may be further fitted into and/or protected by a metallic or aluminum canister.

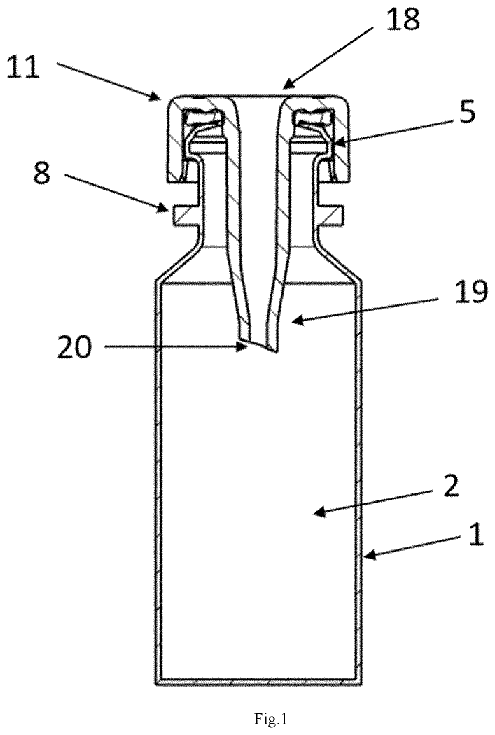

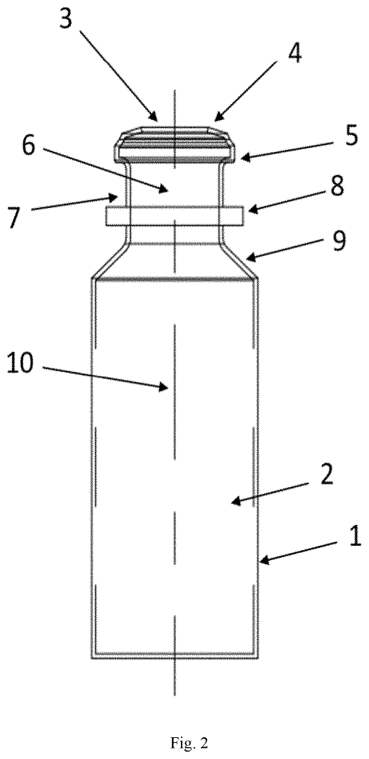

FIG. 1 shows a complete view of the single-layer cartridge comprising the single-layer plastic container and a nozzle shape cap or a stopper. FIG. 2 shows a cross section in the direction of the single-layer plastic container center axis 10, wherein the container wall based on single layer 1 has space 2 for a pharmaceutical formulation solution inside the container. The single-layer cartridge has a cap or stopper lock key 5 projections disposed on the end of the container neck 7 where the single-layer plastic container opening inlet space of cap or stopper 3 nozzle and sealing surface 4 are located. The single-layer plastic container has a transfer bed 8 in between the container neck and slant surface 9 of the container. The transfer bed provides support for the nozzle shaped cap or stopper snap action.

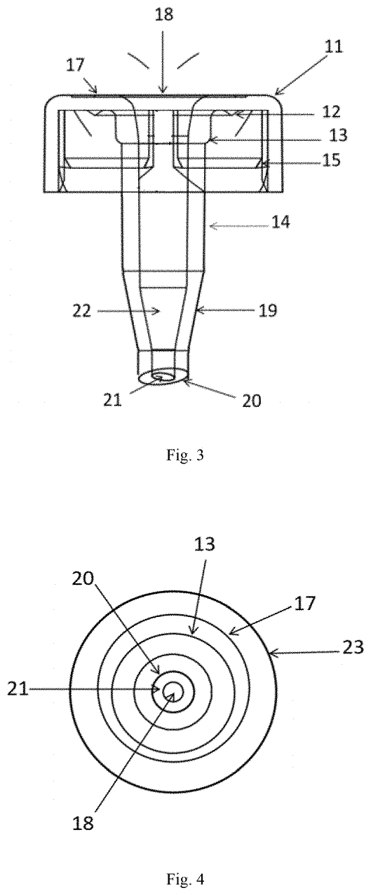

FIG. 3 and FIG. 4 show the nozzle-shaped cap or stopper, wherein the dispensing or soft mist inhalation device or draw-off connection portion has already penetrated the sealing foil on the top side of the top portion 18 and the outlet part 21 of the cap.

The inlet sealing slant provides cannula-free moment space 22, the free moment space 22 penetrates into the neck space insertion connection portion 6 which is sealed with silicon material 20 and is joined to the container sealing surface 4 on its cap or stopper. The stopper is held at its top side by a flanged over portion of the upper end of the stiff casing. The cartridge can be sealed by the stopper with the tight sealing disc 24 which closes to the open end of the capillary tube insertion portion.

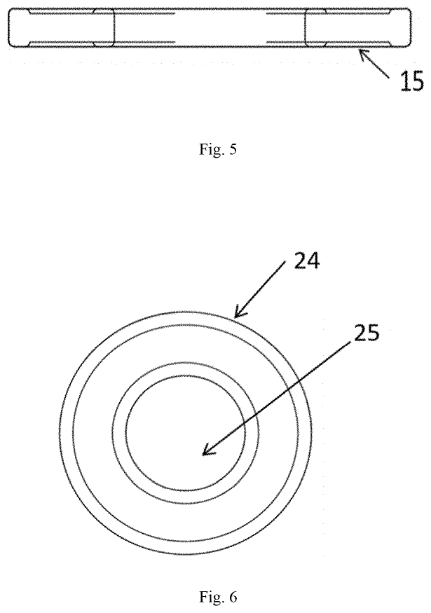

FIG. 5 and FIG. 6 show a sealing disc 24, which is disposed between the upper edge of the container and inlet/outlet space 25 on silicon type material disc inside of the cap or stopper of the cartridge. Disposed between the upper edge sealing surface 4 of the container and the underside of the stopper is the sealing disc 24 having cap locking tab 15. The cap or stopper is held at its top side by a flanged over portion of the upper end of the stiff casing. The cartridge can be sealed by a nozzle-shaped cap or stopper with the tight sealing disc 24 which closes the open end of the capillary tube insertion portion. The cartridge comprises a capillary tube inside a cannula. The capillary breaks the seal of the cap outlet 20 and goes inside the space of container 2.

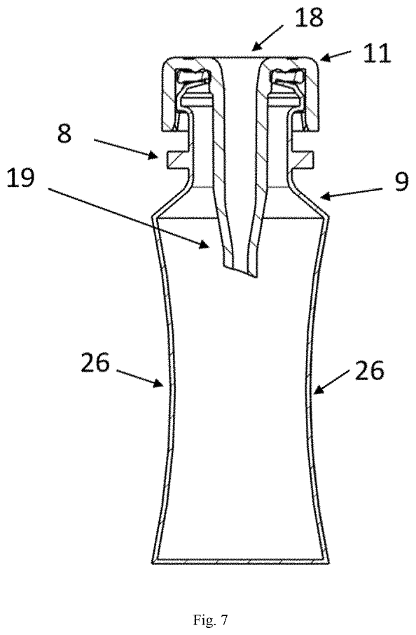

FIG. 7 shows a complete view of a compressed container with the negative pressure effect on the container wall after usage of medicine. When the capillary tube is inserted inside the cartridge, it breaks the seal of the cap or stopper and goes into the cannula while breaking the cap outlet seal 20, and the capillary tube enters inside the container space 2. With the usage of medicine, the container wall 1 is compressed because of the negative pressure. Because the pressure inside the container is lower than the surrounding pressure, this mechanism increases the vacuum inside the container. The compressed negative pressure effect can clearly be seen on the container wall.

The single-layer cartridge according to the current invention can be used with a soft mist inhalation device as shown in FIG. 3 and FIG. 4 of US20190030268 “inhalation atomizer comprising a blocking function and a counter”. The typical inhalable device can be carried anywhere by the patient, since it has a cylindrical shape and a handy size of less than about 8 cm to about 18 cm long, and about 2.5 cm to about 5 cm wide. The nebulizer sprays a defined volume of the pharmaceutical formulation solution out through small nozzles at high pressures, so as to produce an inhalable aerosol.

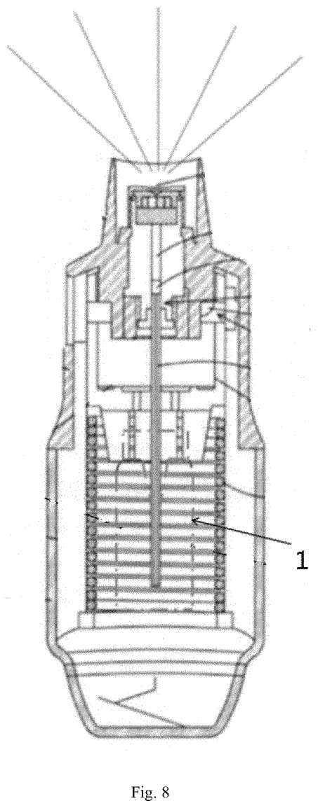

The typical inhalation atomizer comprising the block function and the counter described above for spraying a medicament fluid is depicted in FIG. 8. The inhalation atomizer comprising the block function and the counter described above is preferably a portable inhaler that requires no propellant gas.

The subject of the present invention not only ensures the individual claims, but also the combination of the individual claims. All information and features disclosed in the documentation, including the abstract and, in particular, the structures shown in drawings, are claimed as substantial to the invention insofar as they are novel individually or in combination to the prior art. The innovation is described in more detail with reference to drawings showing several embodiments, features and advantages of significance to the invention which ensue from the drawings.

While various embodiments of the present invention have been described above, it should be understood that they have been presented by way of example only, and not limitation. For example, the present invention is not limited to the physical arrangements or dimensions illustrated or described. Nor is the present invention limited to any particular design or materials of construction. As such, the breadth and scope of the present invention should not be limited to any of the above-described exemplary embodiments, but should be defined only in accordance with the following claims and their equivalents.

### LIST OF REFERENCE NUMERALS USED IN THE FIGURES

1. Single-layer wall plastic container
2. Pharmaceutical formulation solution space inside the container
3. Inlet space of cap or stopper nozzle
4. Sealing surface
5. Cap or stopper lock key
6. Neck space
7. Container Neck
8. Transfer Bed
9. Slant surface
10. Container central axis
11. Upper part Cap with snap-in key
12. Silicon bed hook
13. Outer surface of cap nose
14. Cap or stopper nozzle
15. Cap or stopper locking tab
17. Cap closing space
18. Inlet Opening
19. Inlet sealing slant
20. Cap or stopper outlet Sealing
21. Out let in cap or stopper
22. Cannula free movement space
23. Cap or stopper outer surface
24. Cap Sealing Disc
25. Inlet/outlet space on silicon type material disc
26. Compressed effect

What is claimed is:

1. An inhalation device comprising a cartridge comprising a single-layer plastic container, an opening, and a nozzle-shaped cap or stopper, wherein the cartridge is connected to a soft mist inhalation device.

2. The inhalation device according to claim 1, wherein the nozzle-shaped cap or stopper is connected to the singe-layer container by means of a snap-in connection.

3. The inhalation device according to claim 1, wherein the cartridge further comprises a transfer bed to support the snap-in connection action.

4. The inhalation device according to claim 1, wherein the stopper comprises a plastic material that is non-releasably connected to the single-layer plastic container.

5. The inhalation device according to claim 1, further comprising a sealing disc provided with sealing beads, wherein the sealing disc is disposed between an upper surface of the container and an inside surface of the stopper.

6. The inhalation device according to claim 1, configured such that the cartridge can be pulled out of the soft mist inhalation device by means of a withdrawal aid.

7. The inhalation device according to claim 1 further comprising a rigid outer casing that the cartridge can be inserted into.

8. The inhalation device according to claim 1, further comprising a dispensing device, wherein the dispensing device is provided with snap hook that enables the dispensing device to lock with an upper part of the rigid outer casing after the cartridge has been inserted into the rigid outer casing.

9. The inhalation device according to the claim 1, wherein the single-layer cartridge is connected to the soft mist inhalation device by a push plug-in connection or a bayonet connection.

10. The inhalation device according to claim 1, further comprising a pharmaceutical liquid comprising a pharmaceutically active substance.

11. The inhalation device according to claim 10, wherein the pharmaceutical liquid is selected from the group consisting of water, alcohol, or a combination thereof.

12. The inhalation device according to claim 1, wherein the single-layer plastic container has a thickness of between about 0.05 mm and about 1.0 mm.

13. The inhalation device according to claim 12, wherein the single-layer plastic container has a thickness of between about 0.1 mm and about 0.3 mm.

14. The inhalation device according to claim 1, wherein the single-layer plastic container has a length of between about 20 mm and about 70 mm.

15. The inhalation device according to claim 14, wherein the single-layer plastic container has a length of between about 40 mm and about 55 mm.

16. The inhalation device according to claim 15, wherein the single layer plastic container has a length of between about 45 mm and about 50 mm.

17. The inhalation device according to claim 1, wherein the single layer plastic container is formed by an injection-blow-molding process.
